(19) United States
(12) Plant Patent Application Publication
(10) Pub. No.: US 2013/0340129 P1
(43) Pub. Date: Dec. 19, 2013

(54) Blueberry plant, EB 8-17

(50) ;

(71) Applicant: Vincent David Mazzardis, Joondalup (AU)

(72) Inventor: Vincent David Mazzardis, Joondalup (AU)

(73) Assignees: Rolfe Nominees Pty Ltd., Crows Nest (AU); Prunus Persica Pty Ltd., Joondalup (AU)

(21) Appl. No.: 13/986,861

(22) Filed: Jun. 12, 2013

(30) Foreign Application Priority Data
Jun. 14, 2012 (AU) 2012/114

(51) Int. Cl. A01H 5/00

(52) U.S. Cl. PLT/157

(57) ABSTRACT

A new and distinct variety of blueberry plant, which is denominated varietally hereinafter as ‘EB 8-17’ and which produces a large fruit, which is mature for harvesting and shipment in the early season under the ecological conditions prevailing in Yanchep, Western Australia.

### LATIN NAME

‘Vaccinium Hybrid’

### VARIETAL DENOMINATION

‘EB 8-17’

### RELATED APPLICATION DATA

The present application claims priority from Australian Plant Breeders Rights Application Serial No. 2012/114, and which was filed on 14 Jun. 2012.

### BACKGROUND OF THE NEW VARIETY

The present invention relates to a new, novel, and distinct variety of blueberry plant ‘Vaccinium Hybrid,’ and which has been denominated varietally, hereinafter, as ‘EB 8-17’.

### ORIGIN AND ASEXUAL REPRODUCTION

The present variety of blueberry plant resulted from an ongoing development program which I've conducted to identify new plant varieties. To this end, I make both controlled and hybrid cross-pollinations in order to produce plant populations from which improved progenies are evaluated and thereafter selected.

The blueberry plant ‘EB 8-17’ was originated by me, and selected from a population of new plants growing on my farm, which is located near Yanchep Springs, Yanchep, Western Australia. This new variety of plant was developed in 2005 following the controlled cross-pollination of the seed parent ‘SB-1’ [unpatented] with the pollen parent a blueberry plant named ‘03-6’ [also unpatented]. This first cross-pollination occurred in 2005. The seed parent ‘SB-1’ is characterized by a blueberry plant that has a semi-upright growth pattern, and which further produces large, firm fruit, with an early season of flowering. In contrast, the pollen parent, blueberry plant ‘03-6’ [unpatented] is characterized by a spreading growth habit, and an early season of flowering and a large fruit size. Seeds derived from this original cross-pollination were removed from the seed parent ‘SB-1’ and produced approximately 500 plants. These first plants produced a first crop of fruit in 2007. A further assessment of the fruit produced showed desirable fruit qualities, and growth habits. After conducting a further assessment during the 2008 growing season, the new variety ‘EB 8-17’ was selected as showing particularly desirable traits. Subsequent asexual reproduction of ‘EB 8-17’, by cuttings, occurred during the 2009 through 2011 growing seasons, and led to the conclusion that the blueberry plant ‘EB 8-17’ appeared to be distinct, and novel relative to other known blueberry plants. The present variety of blueberry plant is noteworthy for producing extra large and firm fruit, and which further has a small, dry picking scar and an excellent flavor. The new variety further has early flowering and fruit production dates.

### BRIEF DESCRIPTION OF THE DRAWINGS

The accompanying drawing, which is provided, is a color photograph of the new blueberry plant variety.

FIG. 1 depicts several whole fruit of the new variety of blueberry plant, including mature fruit displaying an external coloration, which is sufficiently matured for harvesting and shipment, as well as several leaves showing the dorsal and ventral coloration thereof, and a vegetative shoot showing the growth habit of the new variety. The supplied photograph further illustrates a shoot bearing typical leaves, and several of the leaves showing the dorsal and ventral coloration thereof, as well as the fruit produced by the closest known variety of blueberry plant named ‘Sharpeblue’ [unpatented].

The colors in these photographs are nearly as true as reasonably possible in a color representation of this type. Due to chemical development, processing and printing, the leaves and fruit depicted in these photographs may or may not be accurate when compared to the actual specimen. For this reason, future color references should be made to the color plates and common color descriptions provided hereinafter.

### NOT A COMMERCIAL WARRANTY

The following detailed description has been prepared to solely comply with the provisions of 35 U.S.C. §112, and does not constitute a commercial warranty (either expressed or implied) that the present variety will, in the future, display the botanical, horticultural or other characteristics as set forth, hereinafter. Therefore, this disclosure may not be relied upon to support any future legal claims including, but not limited to, breach of warranty of merchantability or fitness for any particular purpose, or non-infringement which is directed, in whole, or in part, to the present new variety.

### DETAILED DESCRIPTION

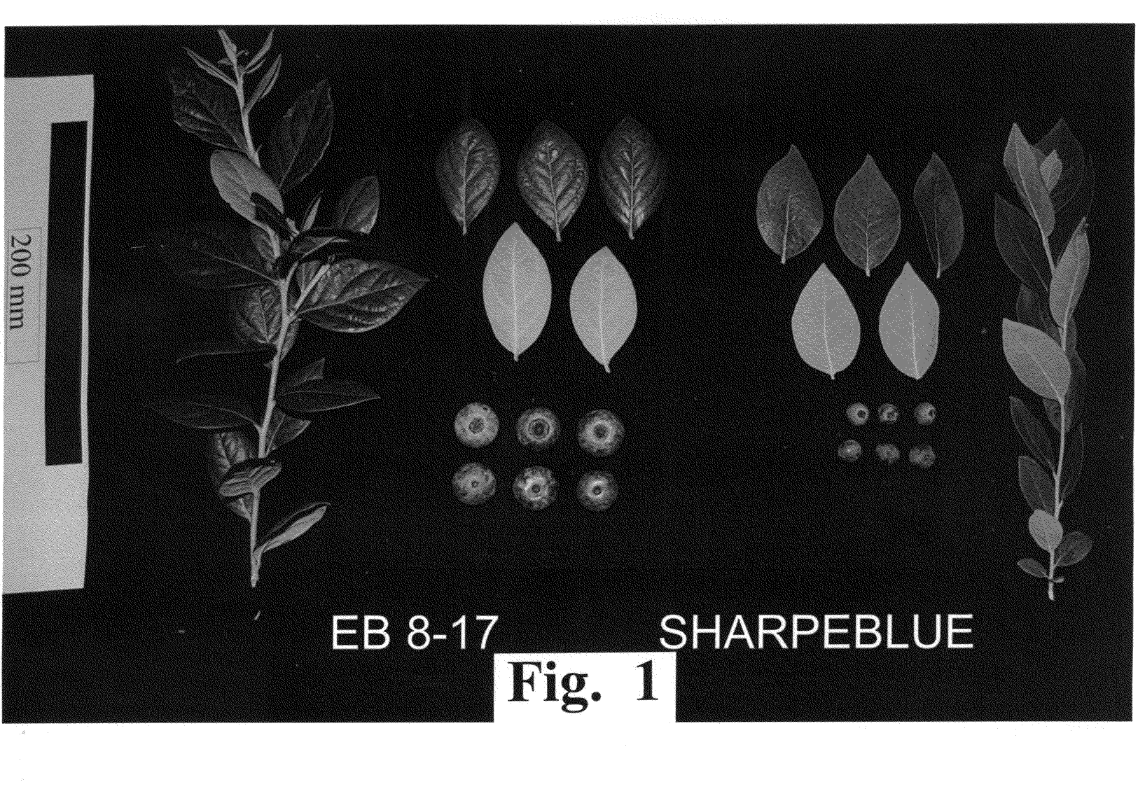

Referring more specifically to the botanical and horticultural details of this new and distinct variety of blueberry plant, the following has been observed during the sixth fruiting season, and under the ecological conditions prevailing at the farm of the inventor, which is located near the town of Yanchep Springs, in Yanchep, Western Australia.
Plant:
   Plant vigor.—Generally — Considered strong for the species. This is in contrast to the vigor as expressed by the ‘Sharpeblue’ blueberry plant where this characteristic is considered only medium to strong.
   Plant growth habit.—Generally — Considered semi-upright. This is in contrast to the growth habit of the ‘Sharpeblue’ blueberry plant which only has an upright growth habit.
   One year old shoots.—Color — Green.
   One year old shoots.—Internode Length — Considered medium to long.
Leaf:
   Leaf length.—Considered medium to long for the species.
   Leaf width.—Considered medium to broad for the species.
   Leaf.—Ratio of Length/Width — Medium to large for the species.
   Leaf shape.—Considered ovate.
   Leaf color.—Dorsal surface — Green.
   Leaf color intensity.—Dorsal surface — Considered dark. This is in contrast to the dorsal surface color as displayed by the ‘Sharpeblue’ blueberry plant where this color is considered light to medium green. This is seen most clearly by reference to FIG. 1.
   Leaf.—Marginal Edge — Entire.
Flower:
   Flower bud coloration.—Generally — A very weak anthocyanin color appears.
   Inflorescence.—Length — Considered average for the species.
   Flower.—Shape — The corolla has an urceolate shape.
   Flower size.—Generally — The corolla has a medium size. This is in contrast to the ‘Sharpeblue’ blueberry plant where this feature of the flower is considered medium to large.
   Corolla tube coloration.—Considered to have a very weak to weak anthocyanin coloration. This is in contrast to the same characteristic in the ‘Sharpeblue’ blueberry plant, which is more predominant, but is still considered weak, overall.
   Flower.—Ridges — Ridges are present on the corolla.
Fruit:
   Fruit cluster density.—Generally — Considered medium for the variety. This is in contrast to the variety most closely similar to it, that being the ‘Sharpeblue’ blueberry plant [unpatented], and which has a fruit cluster density, which is considered to be dense to very dense.
   Unripe fruit color.—Intensity — Considered dark green. This is in contrast to the unripe fruit color intensity of the fruit produced by the ‘Sharpeblue’ blueberry plant, which is the closest known variety, and which displays a light to medium green color.
   Fruit size.—Generally — Considered large for the species. This is in contrast to the fruit produced by the ‘Sharpeblue’ blueberry plant [unpatented], and which produces fruit which is only medium in size. This can be seen most clearly by reference to FIG. 1.
   Fruit shape.—When observed in a longitudinal plane, the fruit is oblate in shape.
   Sepal orientation.—Generally speaking, the sepals are considered semi-erect. This is in contrast to the sepals as displayed in the “Sharpeblue’ blueberry plant where the sepal orientation is considered to be erect.
   Sepal form.—Incurving. This is in contrast to the sepal form as seen in the “Sharpeblue’ blueberry plant where the sepal form is considered to be straight.
   Calyx basin.—Diameter — Considered medium to large. This is in contrast to the fruit produced by the “Sharpeblue’ blueberry plant where the diameter of the calyx basin is considered small to medium for the variety.
   Calyx basin depth.—Considered very shallow, to average in depth.
   Intensity of bloom.—Considered average for the species. This is in contrast to the bloom as seen in the “Sharpeblue’ blueberry plant where this same characteristic is considered to be strong.
   Fruit skin color.—The present color of the skin of the fruit, at full maturity, is considered dark blue.
   Fruit firmness.—Considered medium to firm. This is in contrast to the fruit produced by the ‘Sharpeblue’ blueberry plant, which is considered soft to medium for the species.
   Fruit sweetness.—Considered medium to high for the species. This is in contrast to the fruit sweetness as produced by the ‘Sharpeblue’ blueberry plant, where the sweetness is considered to be only average for the species.
   Fruit.—Acidity — Considered low to medium for the species. This is lower than the average fruit acidity as experienced with the ‘Sharpeblue’ blueberry plant, which is the closest known variety.
   Plant.—Fruiting Type — Fruit appears on one year old, and current season shoots.
   Vegetative bud burst.—Generally — The vegetative bud burst occurs very early in the season. This is earlier in time than the bud burst as observed for the ‘Sharpeblue’ blueberry plant under the same ecological conditions.
   Flowering on one year old shoots.—Generally speaking this date of flowering is quite early in the season, and is earlier in time than the flowering as displayed by the ‘Sharpeblue’ blueberry plant, which is the closest known variety.
   Flowering on current year's shoots.—Generally — Considered very early in the season, and earlier in time than the flowering time as seen on the “Sharpeblue” blueberry plant, which is the closest known variety.
   Date of fruit ripening on one year old shoots.—Considered very early in the season, and earlier in time than the fruit ripening as seen on the ‘Sharpeblue’ blueberry plant, which is the closest known variety, and when grown under similar ecological conditions.
   Fruit on current year's shoots.—Considered very early, and earlier in time than the fruit ripening date for the ‘Sharpeblue’ blueberry plant, which is the closest known variety.
   Resistance to insects and disease.—No particular susceptibilities were noted. The present variety has not been tested to expose or detect any susceptibilities or resistances to any known plant and/or fruit diseases.

Although the new variety of blueberry plant possesses the described characteristics when grown under the ecological conditions prevailing near Yanchep Springs in Yanchep, Western Australia, it should be understood that variations of the usual magnitude and characteristics incident to changes in growing conditions, fertilization, pruning, pest control, frost, climatic variables and horticultural management are to be expected.

Having thus described and illustrated my new variety of blueberry plant, what I claim is new and desire to secure by plant letters patent is:

1. A new and distinct variety of blueberry plant, substantially as illustrated and described, and which is characterized as the novelty by producing a large fruit, which is mature for harvesting and shipment during the early season under the ecological conditions prevailing in Yanchep, Western Australia.
